(19) United States
(12) Patent Application Publication
(10) Pub. No.: US 2023/0264417 A1
(43) Pub. Date: Aug. 24, 2023

(54) 3D BIOPRINTER SYSTEM, SYRINGE ADAPTER, USE OF A 3D BIOPRINTING SYSTEM, AND PROCESS OF EXTRUSION

(71) Applicant: TISSUELABS PESQUISA E DESENVOLVIMENTO LTDA, São Paulo (BR)

(72) Inventors: Emerson Galves MORETTO, São Paulo (BR); Gabriel Romero LIGUORI, São Paulo (BR); Eduardo ZYLBERSTAJN, São Paulo (BR)

(21) Appl. No.: 18/024,650

(22) PCT Filed: Sep. 3, 2021
(86) PCT No.: PCT/BR2021/050378

§ 371 Date: Mar. 3, 2023

(30) Foreign Application Priority Data
Sep. 4, 2020 (BR) 10 2020 018170 0

(51) Int. Cl. B29C 64/209; A61L 27/52; B29C 64/232; B29C 64/245; B29C 64/264; B29C 64/295; B29C 64/336; B29C 64/393

(52) U.S. Cl. CPC B29C 64/209; A61L 27/52; B29C 64/232; B29C 64/245; B29C 64/264; B29C 64/295; B29C 64/336; B29C 64/393; B33Y 10/00

(57) ABSTRACT

The invention relates to the field of additive manufacturing (3D printing) of hydrogels and biological materials. The invention discloses a 3D bioprinting system of multiple hydrogels and biological materials each of which is loaded in a different syringe, using a single extrusion nozzle. It also refers to the syringe adapter, to the uses and applications of the 3D bioprinting system and syringe adapter and to a process for extrusion of hydrogels, biological materials or mixtures thereof.

### FIELD OF THE INVENTION

The present invention relates to the field of additive manufacturing (3D printing) of hydrogels and biological materials.

The present invention discloses a 3D bioprinting system containing a syringe adapter for extruding two or more materials through a single nozzle.

The present invention further discloses the syringe adapter for extruding two or more materials, contained in separate syringes, through a single nozzle.

The present invention further discloses the uses and applications of the 3D bioprinting system and syringe adapter.

The present invention further discloses a process for extrusion of hydrogels, biological materials or mixtures thereof.

### BACKGROUND OF THE INVENTION

3D bioprinting is an additive manufacturing (3D printing) technology in which cells and biomaterials are spatially deposited to fabricate tissue and tissue-like constructs. These constructs can potentially be used in biomedical research and, eventually, for clinical applications. The concept behind the technique is based on the extrusion of hydrogels and biological materials simultaneously to the three-dimensional (X, Y, and Z) movement of the extrusion unit and a receiving platform. The hydrogels and biological materials used in the technique are commonly referred to as bioinks. Bioinks can be either the hydrogels alone or a combination of such hydrogels with cells, constituting a biological material. These hydrogels, in turn, can be synthetic or natural polymers. By combining a choice of hydrogel with a choice of cell, scientists can create a wide range of biological materials to be used in the fabrication of tissue and tissue-like constructs.

One of the most important factors allowing the fabrication of complex tissues, and potentially whole organs, using 3D bioprinting technology, is the ability to combine multiple biological materials within the same construct. The combination of different biological materials is a requirement for fabricating most of the organs and tissues, because the vast majority of them are composed of more than a single cell type. Taking this need in consideration, the use of 3D bioprinters with two or more (multiple) printheads has been proposed and such 3D bioprinters are commercially available. U.S. Patents No. US20180281280A1 and U.S. D861747S1 exemplify 3D bioprinter models with multiple extrusion heads.

The use of multiple extrusion heads, as proposed in the referred patents, for 3D bioprinting complex multi-material tissues and tissue-like structures requires the constant and frequent switch among the different extrusion heads during the 3D printing process. Consequently, it causes interruptions in the 3D construct structure and leads to recurrent deceleration and acceleration for positioning each extrusion head back-and-forth from the 3D construct, reducing print speed and efficiency. Ultimately, it causes increased stress for the cells contained in the biological material.

One proposed alternative for this problem was the use of a microfluidic system containing multiple “micro-jet channels” joining in a single channel with the latter being placed in an extrusion unit, as described in US Patent No. US20160136895A1. The proposed approach, however, requires long tubing systems, which, if presenting a large lumen lead to material loss, and if presenting a thin lumen restrict the use of hydrogels with high viscosity, in addition to requiring the use of small amounts of materials and small volumes. Furthermore, the proposed microfluidic system is driven by pneumatic pressure, limiting the fine-tuning control of the material extrusion.

Another proposed solution was described in Patent No. WO2017184839A1, which discloses a multi-material bioprinter composed of multiple reservoirs connected to an extrusion unit by a similar tubing system as described by US Patent No. US20160136895A1, thus susceptible to the same disadvantages mentioned, besides being designed for in vivo applications. Although it allows the extrusion of greater amounts of materials and volumes in comparison with the microfluidic system, the material loss persists within the system.

In order to overcome the disadvantages of the currently available systems, as described, the present invention discloses a different approach to allow the 3D bioprinting of multiple hydrogels and biological materials each of which is loaded in a different syringe, using a single extrusion nozzle. Since the system is tubeless, and the material is transferred from the syringes directly to the extrusion nozzle by means of the adapters of the invention, the path traveled by the hydrogels with cells is ideal, being as short as possible to reduce shear stress and enhance cell viability. Also, the system of the present invention is a piston-driven system, allowing a fine-tuning control of the material extrusion, as well as a more efficient and faster 3D printing. Finally, the system allows three different types of bioprintings, covering different uses of interest and conferring flexibility to it. The unique combination of features of the 3D bioprinting system of the present invention may be further acknowledged by means of the detailed description below.

### BRIEF SUMMARY OF THE INVENTION

This invention describes improved 3D bioprinters compared to those currently known.

The present invention relates to the field of additive manufacturing (3D printing) of hydrogels and biological materials. It discloses a 3D bioprinting system containing a syringe adapter for extruding two or more materials through a single nozzle. It also discloses the syringe adapter for extruding two or more materials, contained in separate syringes, through a single nozzle and the uses and applications of the 3D bioprinting system and syringe adapter.

The present invention discloses a 3D bioprinting system comprising: a supporting frame; an extrusion unit with two or more dispensing units, each comprising at least: a) a socket where to fit a syringe; wherein the syringe comprises a plunger and a dispensing end; wherein the syringe houses a material to be extruded; and b) a motorized mechanism to move the syringe plunger; a syringe adapter which cooperates with the dispensing end (422) of the two or more syringes and directs the extruded material from the two or more syringes towards a single nozzle; a receiving platform which receives the extruded material from the single nozzle; a mechanical system to independently move the extrusion unit and/or the receiving platform in order to three dimensionaly deposit the extruded material in the receiving platform; and a command unit for controlling the motorized mechanism and the mechanical system.

In a preferred embodiment, the extrusion unit comprises a heating/cooling mechanism, for independently controlling each syringe temperature.

In another preferred embodiment, the extrusion unit comprises one or more lightning systems, wherein the lightning system comprises lights with wavelengths in the ultraviolet, infrared and the visible spectrum.

In another preferred embodiment, the syringe adapter is configured to extrude two or more materials, from two or more syringes, as parallel multi material flow. Preferably, the syringe adapter comprises at least two separate channels.

In another preferred embodiment, the syringe adapter is configured to extrude two or more materials, from two or more syringes, as a homogeneous, mixed, single material flow. Preferably, the syringe adapter comprises at least two separate channels leading to a single mixing chamber. Alternatively, the syringe adapter may comprise a static mixer.

In another preferred embodiment, the syringe adapter is configured to extrude two or more materials, from two or more syringes, as coaxial multi material flow. Preferably, the syringe adapter comprises at least two separate channels, wherein said channels are coaxially disposed.

In another preferred embodiment, the receiving platform includes a heating/cooling mechanism, for controlling the platform surface temperature. Preferably, the receiving platform can dock different adapters for allowing the fitting of diverse depositing surfaces, including Petri dishes, well plates, microplates, glass slides, and beakers.

In another preferred embodiment, the command unit can be controlled by a computerized system, such as a computer connected to the 3D bioprinting system or a touch-screen interface embedded in the 3D bioprinting system.

The present invention also refers to a syringe adapter, designed to be attached to the extrusion unit of the 3D bioprinting system which allows the extrusion of two or more materials, each of them flowing from a different syringe.

Preferably, the syringe adapter can be configured in three different ways. The first, to extrude two or more materials, flowing independently from two or more syringes, as a parallel multi material flow. In this case, the syringe adapter comprises two or more separate channels. The second, to extrude two or more materials, flowing from two or more syringes, as an homogeneous, mixed, single material flow. In this case, the syringe adapter comprises at least two separate channels leading to a single mixing chamber or a static mixer. The third, to extrude two or more materials, flowing from two or more syringes, as a coaxial multi material flow. In this case, the syringe adapter comprises at least two separate channels, wherein said channels are coaxially disposed.

The present invention also refers to a syringe adapter to be docked in a 3D bioprinting system, which cooperates with the dispensing end of two or more syringes and directs an extruded material towards a single nozzle, wherein the syringe adapter comprises: a) at least two separate channels which receive the extruded material from the dispensing ends of the syringes and direct it the single nozzle; or b) at least two separate channels leading to a single mixing chamber or static mixer which receives the extruded material from the dispensing ends of the syringes and directs it the single nozzle; or c) at least two separate channels, wherein said channels are coaxially disposed, which receive the extruded material from the dispensing ends of the syringes and direct it the single nozzle.

The present invention also refers to the use of a 3D bioprinting system, as defined above, for the extrusion of hydrogels, biological materials or mixtures thereof. In a preferred embodiment, the extruded material is used to produce tissue and tissue-like constructs.

The present invention also refers to a process for extrusion of hydrogels, biological materials or mixtures thereof, comprising:

(i) providing a 3D bioprinting system comprising:
a supporting frame;
an extrusion unit with two or more dispensing units, each dispensing unit comprising at least:
(a) a socket where to fit a syringe, wherein the syringe comprise a plunger (421) and a dispensing end (422); wherein the syringe (42) houses a material to be extruded; wherein the material to be extruded comprises hydrogels, biological materials or mixtures thereof;
(b) a motorized mechanism (43) to move the syringe plunger (421) of the syringe (42);
a syringe adapter (5) which cooperates with the dispensing end (422) of the two or more syringes (42) and directs the extruded material from the two or more syringes (42) towards a single nozzle (6);
a receiving platform (7) which receives the extruded material from the single nozzle (6);
a mechanical system (8) to independently move the extrusion unit (3) and/or the receiving platform (7) in order to three dimensionaly deposit the extruded material in the receiving platform (7);
a command unit (9) for controlling the motorized mechanism (43) and the mechanical system (8);
   (ii) fitting two or more syringes (42) into the two or more sockets (41) provided in the extrusion unit (3);
   (iii) providing instructions to the command unit (9);
   (iv) dispensing the hydrogels, biological materials or mixtures thereof from the syringes (42) into the syringe adapter (5);
   (v) dispensing the hydrogels, biological materials or mixtures thereof from the syringe adapter (5) through the single nozzle (6);
   (vi) extruding the hydrogels, biological materials or mixtures thereof into the receiving platform (7).

### BRIEF DESCRIPTION OF THE DRAWINGS

FIG. 1 represents a schematic perspective view of one embodiment of the 3D bioprinting system described herein.

FIG. 2 discloses an example of one embodiment of the supporting frame described herein.

FIG. 3 discloses an example of one embodiment of the extrusion unit described herein.

FIGS. 4a to 4d disclose examples of some embodiments of the syringe adapter described herein.

FIG. 5 discloses an example of one embodiment of the receiving platform described herein.

FIG. 6 discloses an example of one embodiment of the mechanical system described herein.

FIG. 7 discloses an example of one embodiment of the command unit described herein.

### DETAILED DESCRIPTION

The present invention can be more readily understood considering the detailed description, figures, and examples described and/or shown in this document. The possible embodiments of this invention, however, are not limited to the specific devices and setups described herein, nor are the methods and applications of this invention limited to those herein detailed.

It is to be understood that features herein described as separate entities may also be provided in combination. Oppositely, different features herein described as a single entity may also be provided separately.

Throughout this document, the expression “at least one” is equivalent to the expression “one or more”, and means one or more members, or at least one member of a group of members. These are terms that include any of ≥1, ≥2, ≥3, ≥4, ≥5, ≥6, ≥7, etc. of said members, and even all said members (for instance, 1 to 2, 1 to 3, and so on). The same reasoning can be understood for the expression “at least two”, and so on.

Throughout this document, words and expressions such as “preferentially”, “particularly”, “for example”, “such as”, “more particularly”, “more preferably” and the like, and their variations, should be interpreted as entirely optional characteristics, preferential embodiments or possible non-exhaustive examples, without giving a limiting scope.

Throughout this document, the word “comprises”, and its variations, such as “comprise” or “comprising”, should be interpreted as “open terms”, which may include additional elements or groups of elements, which were not explicitly mentioned or described, not having a limiting character. The same should be understood for the words “includes” and “contains”, and their variations.

Throughout this document, the word “consists”, and any variations such as “consist” or “consisting”, should be interpreted as “closed terms”, and may not imply the inclusion of additional elements or groups of elements, which have not been explicitly described, having a limiting character.

When not explicitly stated otherwise, all acronyms, expressions and/or technical terms are to be interpreted as having the meanings generally used and widely known in the technical field of the present invention. In some cases, terms with commonly understood meanings are defined in this document for the purpose of clarity and/or for prompt reference, and the inclusion of such definitions in this document should not necessarily be interpreted as representing a substantial difference in what is generally understood in the prior art.

The present invention relates to the field of additive manufacturing (3D printing) of hydrogels and biological materials. It discloses a 3D bioprinting system containing a syringe adapter for extruding two or more materials through a single nozzle. It also discloses the syringe adapter for extruding two or more materials, contained in separate syringes, through a single nozzle and the uses and applications of the 3D bioprinting system and syringe adapter.

The present invention discloses a 3D bioprinting system 1 comprising: a supporting frame 2; an extrusion unit 3 with two or more dispensing units 4, each dispensing unit 4 comprising at least: a) a socket 41 where to fit a syringe 42, wherein the syringe 42 comprises a plunger 421 and a dispensing end 422; wherein the syringe 42 houses a material to be extruded; and b) a motorized mechanism 43 to move the syringe plunger 421; a syringe adapter 5 which cooperates with the dispensing end 422 of the two or more syringes and directs the extruded material from the two or more multiple syringes 42 towards a single nozzle 6; a receiving platform 7 which receives the extruded material from the single nozzle 6; a mechanical system 8 to independently move the extrusion unit 3 and the receiving platform 7 in order to three dimensionaly deposit the extruded material in the receiving platform 7; and a command unit 9 for controlling the motorized mechanism 43 and the mechanical system 8, therefore underlying the movements and function of the extrusion unit 3 and receiving platform 7.

In accordance with the present invention, the material to be extruded preferably comprises hydrogels, biological materials or mixtures thereof.

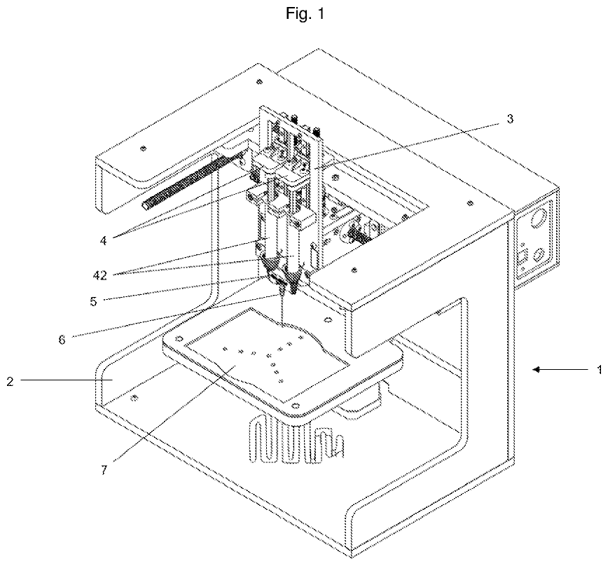

FIG. 1 discloses an example of one embodiment of the 3D bioprinting system described herein.

The supporting frame 2 is designed to sustain the extrusion unit 3, the receiving platform 7, the mechanical system 8, and the command unit 9. Not all these parts must be directly sustained by the supporting frame 2, being possible that any of them is sustained by another part which, in turn, is sustained by the supporting frame 2.

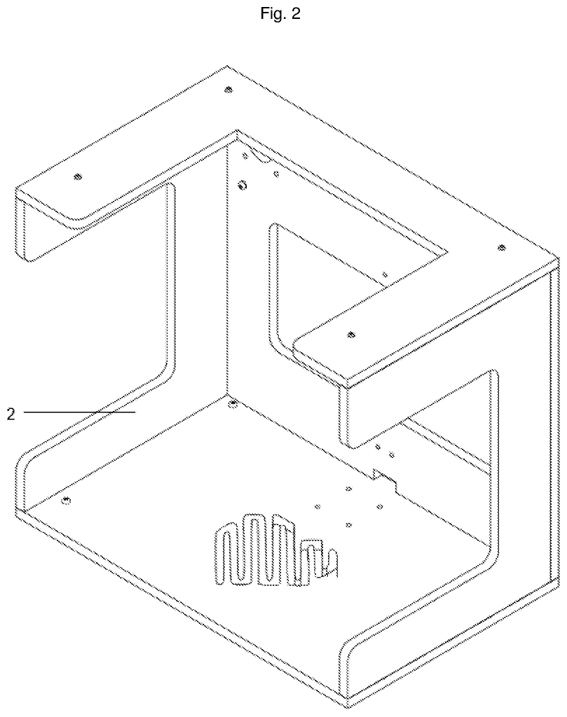

FIG. 2 discloses an example of one embodiment of the supporting frame described herein.

The extrusion unit 3 is composed of two or more dispensing units 4, each comprising at least a socket 41 where to fit a syringe 42 and a motorized mechanism 43 to move the syringe plunger 421. In a preferred embodiment, the socket 41 to fit each syringe 42 contains at least one supporting spot. More preferably, there are two supporting spots; the first spot attaches to the syringe in the region designed for needle attachment and the other attaches to the barrel flanges.

Alternative designs are possible, but the herein described setup is preferable. For each dispensing unit 4, in a preferred embodiment, the motorized mechanism 43 comprises a set of at least one motor 431 and guiding mechanism 432. Each motor 431 can be a stepper motor containing a lead screw 433 or, alternatively, have a lead screw attached to it with a coupler. To each lead screw 433, at least one sliding feed nut 434 can be attached. The sliding feed nut 434 can be connected, directly or indirectly, to the syringe plunger 421 flange. The syringe plunger 421 flange can be connected to a guiding mechanism 432 that can be formed of one or more rods 435, or one or more linear guides, one or more belts, or any combination of them.

Alternatively, the extrusion unit 3 may comprise a single motor 431, which actuates all the dispensing units 4 through several connected arms.

Each dispensing unit 4 can be independently controlled by the command unit 9 so that each syringe 42 can extrude at different flow rates, being possible that only one syringe 42 is used at a time or that two or more syringes 42 are used simultaneously.

Each dispensing unit 4 may include a heating or a cooling mechanism, or both, so that each syringe 42 temperature can be independently controlled. Temperature in each syringe 42 can be controlled to remain in any specific value between 4° C. and 250° C.

Each dispensing unit 4 may include one or more a lightning system, which can be used for photocrosslinking of the extruded material, wherein the lightning system comprise lights with wavelengths in the ultraviolet, infrared and the visible spectrum.

Photocrosslinking systems known in the art usually operate with wavelengths at 257, 365, 385, 395, or 405 nm. Alternatively, systems operating between 400-450 nm, at approximately 500 nm, and also at the infrared spectrum can be used.

The extrusion unit 3 may be fixed (in any embodiment in which the receiving platform 7 moves in the three axes), or it can move in a single axis, or it can move in two axes, or it can move in all the three axes. In a preferred embodiment, the receiving platform 7 should move exclusively in the X and Y axis.

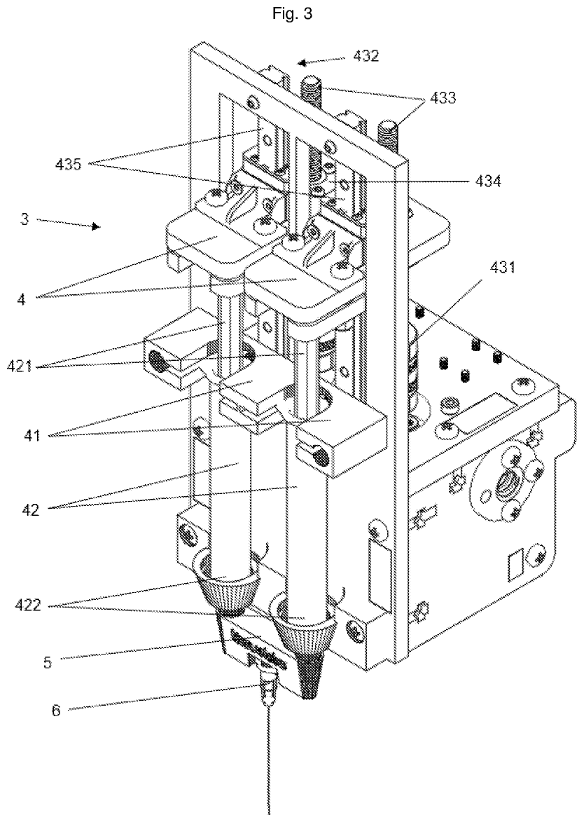

FIG. 3 discloses an example of one embodiment of the extrusion unit described herein.

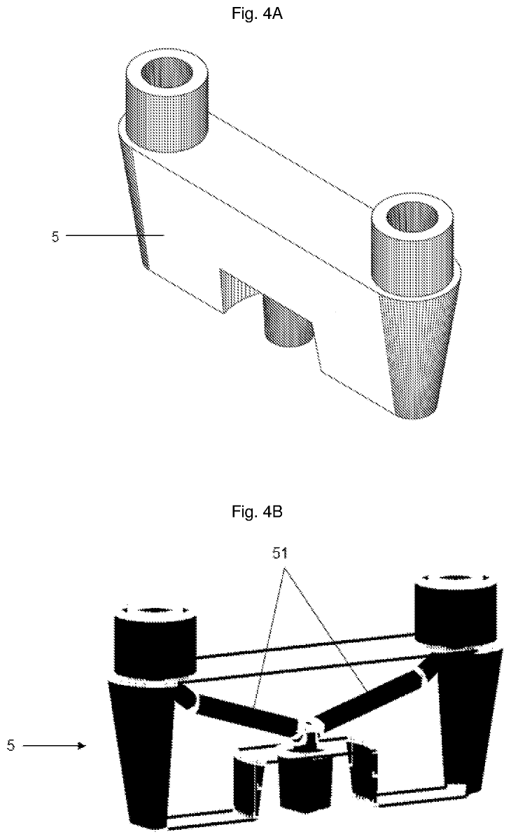

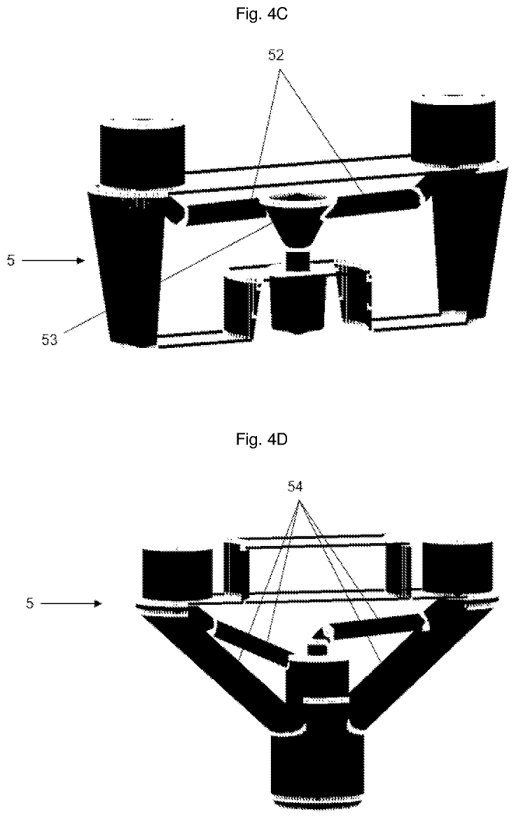

The syringe adapter 5 is designed to be attached to the extrusion unit 3 of a 3D bioprinting system 1. The syringe adapter 5 receives the extruded material by cooperating with the dispensing end 422 of two or more syringes 42 and directs all the material flow from these multiple syringes 42, towards a single nozzle 6, as shown in FIG. 4a.

In a preferred embodiment, the syringe adapter 5 can be composed by two or more channels, each one leaving from a different syringe 42, and all connecting to a single mixing chamber or static mixer, or to multiple coaxial channels, which are ultimately connected in a single nozzle 6.

The syringe adapter 5 can be designed in numerous shapes to extrude multiple materials in diverse methods. Herein three preferable ways the adapter can be designed are described.

In the first preferred embodiment, two or more materials, flowing from two or more syringes 42, are extruded by a single nozzle 6 as a parallel multi material flow. In this embodiment, at least two separate channels 51 are defined inside the syringe adapter 5, as shown in FIG. 4b.

In the second preferred embodiment, two or more materials, flowing from two or more syringes 42, are extruded by a single nozzle 6 as a homogeneous, mixed, single material flow. In this embodiment, at least two separate channels 52 leading to a single mixing chamber 53 or static mixer are defined inside the syringe adapter 5, as shown in FIG. 4c.

In the third preferred embodiment, two or more materials, flowing from two or more syringes 42, are extruded by a single nozzle 6 as a coaxial multi material flow. In this embodiment, at least two separate channels 54 are defined inside the syringe adapter 5, wherein said channels 54 are coaxially disposed, as shown in FIG. 4d.

The latter is particularly useful in case of materials that have to be chemically cross-linked or for the fabrication of tubular structures. Regardless of the three mentioned embodiments and the number of syringes, the material(s) do be extruded(s) is directed to the same nozzle

In a preferred embodiment, the syringes 42 are commercially available syringes (3, 5, and 10 mL) known in the state of the art, with no particular features to cooperate to the syringe adapter 5 and the motorized mechanism 43. This represent a further advantage in view of the prior-art documents, which disclose several specific reservoirs for receiving the material to be extruded, as well as long channels used to dispense the material.

The solutions disclosed in the prior-art documents do not allow the use of commercially available syringes, and therefore require complex structures which negatively impact the extrusion process as a whole, as previously stated.

The receiving platform 7 which receives the extruded material is preferably composed of a flat surface connected to the supporting frame 2 either directly or indirectly, in case it is connected solely to the mechanical system 8.

The receiving platform 7 of the 3D bioprinting system 1 may include a heating or a cooling mechanism, or both, for controlling the platform surface temperature.

The receiving platform 7 can dock different exchangeable adapters for allowing the fitting of diverse depositing surfaces, including Petri dishes, well plates, microplates, glass slides, and beakers. The receiving platform 7 may be fixed (in any embodiment in which the extrusion unit 3 moves in the three axes), or it can move in a single axis, or it can move in two axes, or it can move in all the three axes. In a preferred embodiment the receiving platform 7 should move exclusively in the Z axis.

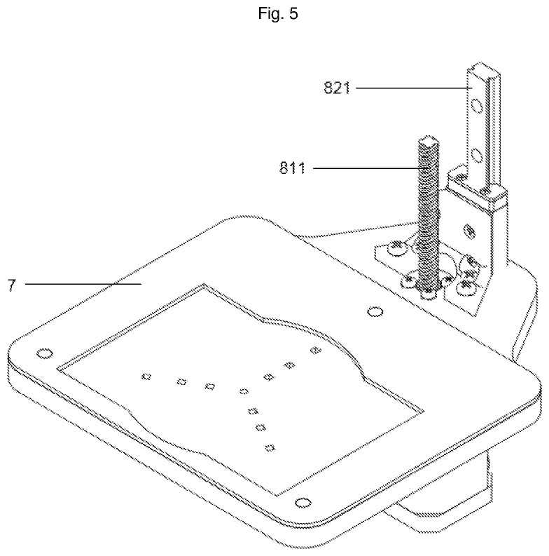

FIG. 5 discloses an example of one embodiment of the receiving platform described herein.

The mechanical system 8 designed to independently move the extrusion unit 3 and/or the receiving platform 7 is composed of three axes (X, Y, and Z). In a preferred embodiment, the mechanical system 8 comprises a set of at least one motor 81 and guiding mechanism 82 for each of the axes. Each motor 81 can be a stepper motor containing a lead screw 811 or, alternatively, have a lead screw attached to it with a coupler. To each lead screw 811, at least one sliding feed nut 812 can be attached. The sliding feed nut 812 can be connected, directly or indirectly, to the extrusion unit 3 or to the receiving platform 7 designed for receiving the extruded material. The extrusion unit 3 and/or the receiving platform 7 can be connected to a guiding mechanism 82 that can be formed of one or more rods 821, or one or more linear guides, one or more belts, or any combination of them. The three sets motor 81 and guiding mechanism 82 (X, Y, and Z), can all be connected exclusively to the extrusion unit 3, or exclusively the receiving platform 7. Alternatively, two of the sets can be attached to the extrusion unit 3 and one to the receiving platform 7, or, still, two of the sets can be attached to the receiving platform 7 and one to the extrusion unit 3.

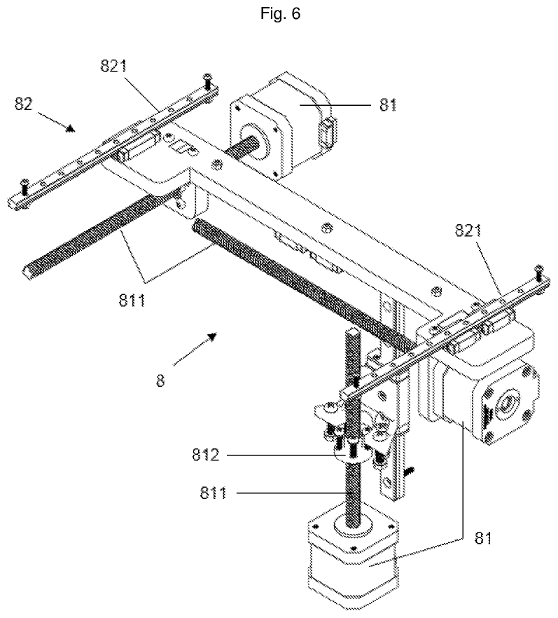

FIG. 6 discloses an example of one embodiment of the mechanical system described herein.

The command unit 9 is responsible for controlling the mechanisms underlying the movements and function of the extrusion unit 3 and receiving platform 7. The command unit 9 comprises at least one control board 91, at least one microcontroller or microprocessor, and one or more control drivers 92. This unit is responsible for interpreting and effectuating the commands coming from a computer, and/or mobile device, and/or memory card. The connection with the computer or mobile device can be wired or wireless and the connection with the memory card can be direct or indirect.

Among the possible commands received and effectuated by the command unit 9 are the movements of the mechanical system 8 and the operation of the extrusion unit 3, including not only the motorized mechanism 43, but also the control of temperature and lighting.

The command unit 9 of the 3D bioprinting system 1 can be controlled either by a computerized system, such as a computer or mobile device connected to the 3D bioprinting system 1, wired or wireless—or by an interface embedded in the 3D bioprinting system 1.

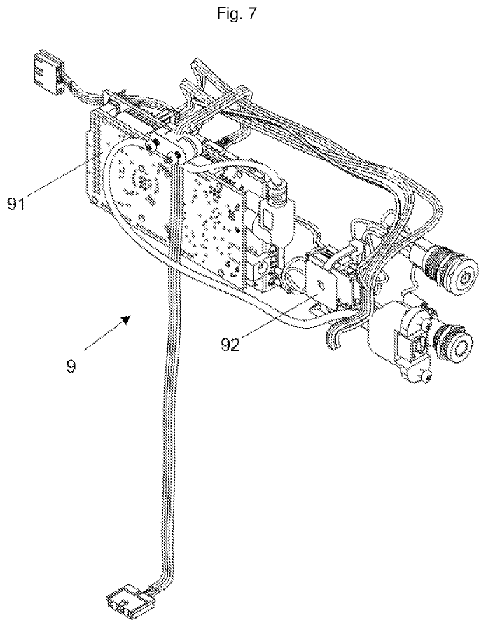

FIG. 7 discloses an example of one embodiment of the command unit described herein.

The present invention also refers to a syringe adapter 5 to be docked in a 3D bioprinting system 1, which cooperates with the dispensing end 422 of two or more syringes 42 and directs an extruded material towards a single nozzle 6.

More specifically, the syringe adapter 5 comprises:
(a) at least two separate channels 51 which receive the extruded material from the dispensing ends 422 of the syringes 42 and direct it the single nozzle 6; or
(b) at least two separate channels 52 leading to a single mixing chamber 53 or static mixer which receives the extruded material from the dispensing ends 422 of the syringes 42 and directs it the single nozzle 6; or
(c) at least two separate channels 54, wherein said channels 54 are coaxially disposed, which receive the extruded material from the dispensing ends 422 of the syringes 42 and direct it the single nozzle 6.

The syringe adaptor of the present invention may be used with the 3D bioprinting system as described, or may be used with different 3D bioprinting systems available or to be configured.

The present invention also refers to the use of a 3D bioprinting system 1, as defined above, for the extrusion of hydrogels, biological materials or mixtures thereof. Preferably, the extruded material is used to produce tissue and tissue-like constructs.

The present invention also refers to a process for extrusion of hydrogels, biological materials or mixtures thereof, comprising the steps of:

(i) providing a 3D bioprinting system 1 comprising:
a supporting frame 2;
an extrusion unit 3 with two or more dispensing units 4, each dispensing unit 4 comprising at least:
(a) a socket 41 where to fit a syringe 42, wherein the syringe 42 comprise a plunger 421 and a dispensing end 422; wherein the syringe 42 houses a material to be extruded; wherein the material to be extruded comprises hydrogels, biological materials or mixtures thereof;
(b) a motorized mechanism 43 to move the syringe plunger 421 of syringe 42;
a syringe adapter 5 which cooperates with the dispensing end 422 of the two or more syringes 42 and directs the extruded material from the two or more syringes 42 towards a single nozzle 6;
a receiving platform 7 which receives the extruded material from the single nozzle 6;
a mechanical system 8 to independently move the extrusion unit 3 and/or the receiving platform 7 in order to three dimensionaly deposit the extruded material in the receiving platform 7;
a command unit 9 for controlling the motorized mechanism 43 and the mechanical system 8;
   (ii) fitting two or more syringes 42 into the two or more sockets 41 provided in the extrusion unit 3;
   (iii) providing instructions to the command unit 9;
   (iv) dispensing the hydrogels, biological materials or mixtures thereof from the syringes 42 into the syringe adapter 5;
   (v) dispensing the hydrogels, biological materials or mixtures thereof from the syringe adapter 5 through the single nozzle 6;
   (vi) extruding the hydrogels, biological materials or mixtures thereof into the receiving platform 7.

1. A 3D bioprinting system (1) comprising:
a supporting frame (2);
an extrusion unit (3) with two or more dispensing units (4), each dispensing unit (4) comprising at least:
(a) a socket (41) where to fit a syringe (42), wherein the syringe (42) comprises a plunger (421) and a dispensing end (422); wherein the syringe (42) houses a material to be extruded;
(b) a motorized mechanism (43) to move the syringe plunger (421) of syringe (42);
a syringe adapter (5) which cooperates with the dispensing end (422) of the two or more syringes (42) and directs the extruded material from the two or more syringes (42) towards a single nozzle (6);
a receiving platform (7) which receives the extruded material from the single nozzle (6);
a mechanical system (8) to independently move the extrusion unit (3) and/or the receiving platform (7) in order to three dimensionaly deposit the extruded material in the receiving platform (7);
a command unit (9) for controlling the motorized mechanism (43) and the mechanical system (8).

2. A 3D bioprinting system according to claim 1, wherein the extrusion unit (3) comprises a heating/cooling mechanism for independently controlling each syringe (42) temperature.

3. A 3D bioprinting system according to claim 1, wherein the extrusion unit (3) comprises one or more lightning systems, wherein the lightning system comprises lights with wavelengths in the ultraviolet, infrared and the visible spectrum.

4. A 3D bioprinting system according to claim 1, wherein the syringe adapter (5) is configured to extrude two or more materials, from two or more syringes (42), as parallel multi material flow.

5. A 3D bioprinting system, according to claim 4, wherein the syringe adapter (5) comprises at least two separate channels (51).

6. A 3D bioprinting system according to claim 1, wherein the syringe adapter (5) is configured to extrude two or more materials, from two or more syringes (42), as a homogeneous, mixed, single material flow.

7. A 3D bioprinting system, according to claim 6, wherein the syringe adapter (5) comprises at least two separate channels (52) leading to a single mixing chamber (53) or static mixer.

8. A 3D bioprinting system according to claim 1, wherein the syringe adapter (5) is configured to extrude two or more materials, from two or more syringes (42), as coaxial multi material flow.

9. A 3D bioprinting system, according to claim 8, wherein the syringe adapter (5) comprises at least two separate channels (54), wherein said channels (54) are coaxially disposed.

10. A 3D bioprinting system according to claim 1, wherein the receiving platform (7) includes a heating/cooling mechanism for controlling the platform surface temperature.

11. A 3D bioprinting system according to claim 1, wherein the receiving platform (7) can dock different adapters for allowing the fitting of diverse depositing surfaces, including Petri dishes, well plates, microplates, glass slides, and beakers.

12. A 3D bioprinting system according to claim 1, wherein the command unit (9) can be controlled by a computerized system, such as a computer connected to the 3D bioprinting system or a touch-screen interface embedded in the 3D bioprinting system.

13. A syringe adapter (5) to be docked in a 3D bioprinting system (1), which cooperates with the dispensing end (422) of two or more syringes (42) and directs an extruded material towards a single nozzle (6), wherein the syringe adapter (5) comprises:
(a) at least two separate channels (51) which receive the extruded material from the dispensing ends (422) of the syringes (42) and direct it the single nozzle (6); or
(b) at least two separate channels (52) leading to a single mixing chamber (53) or static mixer which receives the extruded material from the dispensing ends (422) of the syringes (42) and directs it the single nozzle (6); or
(c) at least two separate channels (54), wherein said channels (54) are coaxially disposed, which receive the extruded material from the dispensing ends (422) of the syringes (42) and direct it the single nozzle (6).

14. A syringe adapter according to claim 13, wherein the syringe adapter (5) is configured to extrude two or more materials, from two or more syringes (42), as parallel multi material flow.

15. A syringe adapter according to claim 13, wherein the syringe adapter (5) is configured to extrude two or more materials, from two or more syringes (42), as an homogeneous, mixed, single material flow.

16. A syringe adapter according to claim 13, wherein the syringe adapter (5) is configured to extrude two or more materials (42), from two or more syringes, as coaxial multi material flow.

17. (canceled)

18. (canceled)

19. A process for extrusion of hydrogels, biological materials or mixtures thereof, comprising:
(i) providing a 3D bioprinting system (1) comprising:
a supporting frame (2);
an extrusion unit (3) with two or more dispensing units (4), each dispensing unit (4) comprising at least:
(a) a socket (41) where to fit a syringe (42), wherein the syringe (42) comprise a plunger (421) and a dispensing end (422); wherein the syringe (42) houses a material to be extruded; wherein the material to be extruded comprises hydrogels, biological materials or mixtures thereof;
(b) a motorized mechanism (43) to move the syringe plunger (421) of syringe (42);
a syringe adapter (5) which cooperates with the dispensing end (422) of the two or more syringes (42) and directs the extruded material from the two or more syringes (42) towards a single nozzle (6);
a receiving platform (7) which receives the extruded material from the single nozzle (6);
a mechanical system (8) to independently move the extrusion unit (3) and/or the receiving platform (7) in order to three dimensionaly deposit the extruded material in the receiving platform (7);
a command unit (9) for controlling the motorized mechanism (43) and the mechanical system (8);
(ii) fitting two or more syringes (42) into the two or more sockets (41) provided in the extrusion unit (3);
(iii) providing instructions to the command unit (9);
(iv) dispensing the hydrogels, biological materials or mixtures thereof from the syringes (42) into the syringe adapter (5);
(v) dispensing the hydrogels, biological materials or mixtures thereof from the syringe adapter (5) through the single nozzle (6);
(vi) extruding the hydrogels, biological materials or mixtures thereof into the receiving platform (7).
